(19) United States
(12) Patent Application Publication
(10) Pub. No.: US 2005/0197642 A1
(43) Pub. Date: Sep. 8, 2005

(54) Buckling device for disposable articles

(75) Inventor: Sen-Mei Cheng, Changhua Hsien (TW)

Correspondence Address: TROXELL LAW OFFICE PLLC, 5205 Leesburg Pike,, Suite 1404, Falls Church, VA, 22041, US

(73) Assignee: Taiwan Paiho Limited

(21) Appl. No.: 10/790,807

(22) Filed: Mar. 3, 2004

(51) Int. Cl. A61F 13/15

(52) U.S. Cl. 604/391

(57) ABSTRACT

A buckling device for disposable articles including a disposable article made up of a front piece with a fleece sticker, and a rear piece having adjusting buckle belts attached at both opposing lateral edges to be mutually fastened to the fleece sticker. The adjusting buckle belt has a buckling section made up of a plurality of jump-type fastening facets and flexible soft facets alternatively arranged at the inner lateral side to keep the proper softness of the buckling section. Thus, in buckling engagement, the fastening facets of the buckling sections will be firmly attached to the fleece sticker respectively in a jump-type manner with the flexible soft facets alternatively dividing the fastening facets so that both the front and rear parts of the buckling sections can be precisely buckled up with the fleece sticker preventing the buckling sections from being twisted or torn apart easily.

### BACKGROUND OF THE INVENTION

The present invention is related to a buckling device for disposable articles, including a fleece sticker disposed at a front piece thereon, and adjusting buckle belts attached at both lateral edges of a rear piece thereon to be fastened to the fleece sticker thereby wherein the adjusting buckle belt has a buckling section made up of a plurality of jump-type fastening facets and flexible soft facets alternatively arranged at the inner lateral side thereon to keep the proper softness of the buckling section thereof; whereby, in buckling engagement, the fastening facets of the buckling sections will be firmly attached to the fleece sticker respectively in a jump-type manner so that both the front and rear parts of the buckling sections thereof can be precisely buckled up with the fleece sticker thereof to effect the best buckle engagement thereof, preventing the buckling sections thereof from being twisted or torn apart easily by toddlers in great and active movements. Besides, via the flexible soft facets alternatively arranged with the fastening facets thereof, the buckling sections are kept in proper softness as a whole, permitting a comfortable feeling to the toddlers by the belly skin while walking around with the disposable article thereof so as to achieve the best using condition thereof.

Please refer to FIG. 1. A conventional disposable article 10 is made up of a rear piece 11 having adjusting buckle belts 12 attached at both lateral edges thereon to be mutually fastened to a fleece sticker 14 disposed at a front piece 13 thereon. The adjusting buckle belt 12 has a fastening section 121 disposed at one end thereon for registration with the fleece sticker 14 thereby, and a soft section 122 disposed at one side of the buckling section 121 adjacent to the lateral edge of the rear piece 11 thereof.

Please refer to FIG. 2. In buckling engagement, the fastening sections 121 of the adjusting buckle belts 12 are mutually registered with the fleece sticker 14 thereof. In case that the conventional disposable article 10 is worn by toddlers in great and active movements, the soft sections 122 of the adjusting buckle belts 12 are easily turned outwards and make the fastening sections 121 torn apart therewith. Thus, the conventional disposable article 10 can easily come off as a whole in practical use.

Please refer to FIG. 3. Another conventional disposable article 10′ is equipped with a rear piece 11′ having adjusting buckle belts 12′ attached at both lateral edges thereon wherein the adjusting buckle belt 12′ has an extending buckle section 121′ integrally disposed at one end thereon to be registered with a fleece sticker 14′ of a rear piece 13′ thereby.

There are some drawbacks to the second conventional disposable article 10′. First, the extending buckle sections 121′ of the adjusting buckle belt 12′, though effecting a better buckling engagement thereof, tend to form a hard texture when firmly fastened to the fleece sticker 14′ thereof, which can result in uncomfortable allergy such as pain or swollen belly skin to toddlers when walking around with the disposable article 10′ thereof. Second, the extending buckle sections 121′ firmly fastened to the fleece sticker 14′, are hard to be detached there-from for replacement thereof. The sudden noisy sound of continuing tearing off of the adjusting buckle belts 12′ to detach the extending buckle sections 121′ from the fleece stickers 14′ thereof can easily awake the toddlers in sound asleep. Third, despite of the better buckling effect thereof, the extending buckle sections 121′ will increase the cost of materials and boost the price of sale therewith, which makes the second conventional disposable article uncompetitive in the market.

### SUMMARY OF THE PRESENT INVENTION

It is, therefore, the primary purpose of the present invention to provide a buckling device for disposable articles, including adjusting buckle belts attached at both lateral edges of a rear piece thereon to be mutually fastened to a fleece sticker of a front piece thereby wherein the adjusting buckle belt has a buckling section made up of a plurality of jump-type fastening facets and flexible soft facets alternatively arranged at the inner lateral side thereon to keep the proper softness of the buckling section thereof; whereby, in buckling engagement, the fastening facets of the buckling sections will be firmly attached to the fleece sticker thereof respectively in a jump-type manner so that both the front and rear parts of the buckling sections thereof can be precisely buckled up with the fleece sticker thereof to effect the best buckle engagement thereof, preventing the buckling sections thereof from being twisted or torn apart easily by toddlers in great and active movements.

It is, therefore, the second purpose of the present invention to provide a buckling device for disposable articles wherein, via the flexible soft facets alternatively arranged with the fastening facets thereof, the buckling sections are kept in proper softness as a whole, permitting a comfortable feeling to the toddlers by the belly skin while walking around with the disposable article so as to achieve the best using condition thereof.

It is, therefore, the third purpose of the present invention to provide a buckling device for disposable articles wherein the fastening facets of the buckling sections with the flexible soft sections alternatively arranged there-between are firmly attached to the fleece sticker thereof respectively in a jump-type manner so that the tearing sound of the adjusting buckle belts to detach the fastening sections from the fleece sticker for replacement thereof will fall in steps to avoid waking up the toddlers in sound asleep.

### BRIEF DESCRIPTION OF THE DRAWINGS

FIG. 1 is a perspective view of a conventional disposable article.

FIG. 2 is a diagram showing the coming off of an adjusting buckle belt of the conventional disposable article.

FIG. 3 is a perspective view of another disposable article.

FIG. 4 is a perspective view of the present invention.

FIG. 5 is a diagram showing the present invention in practical use.

FIG. 6 is a cross sectional view of the present invention in practical use.

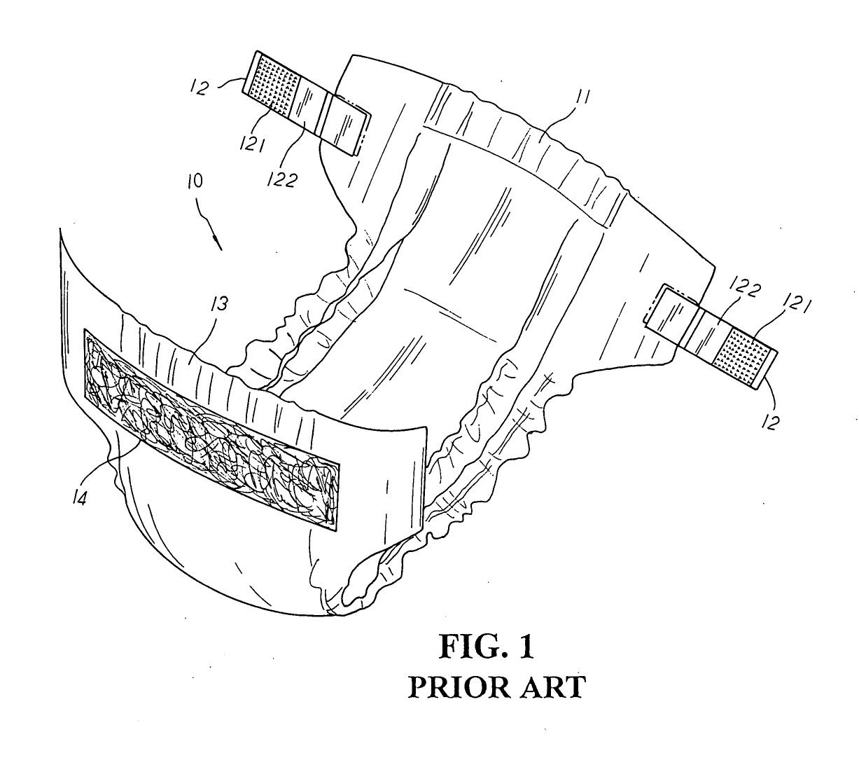

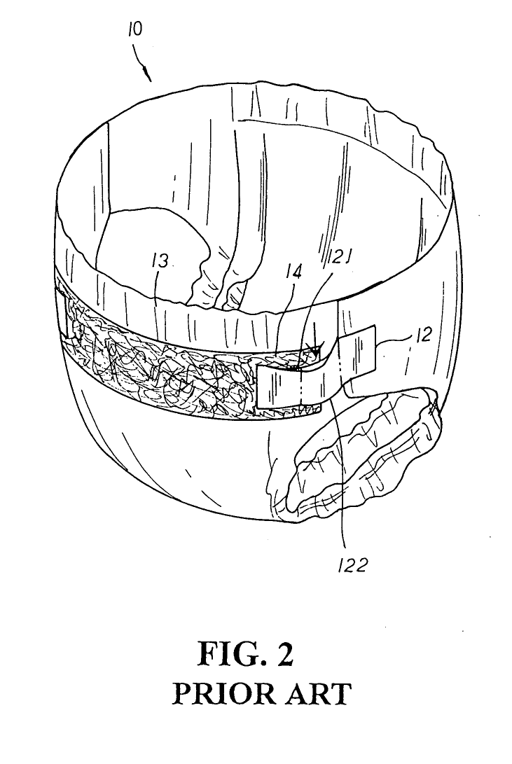

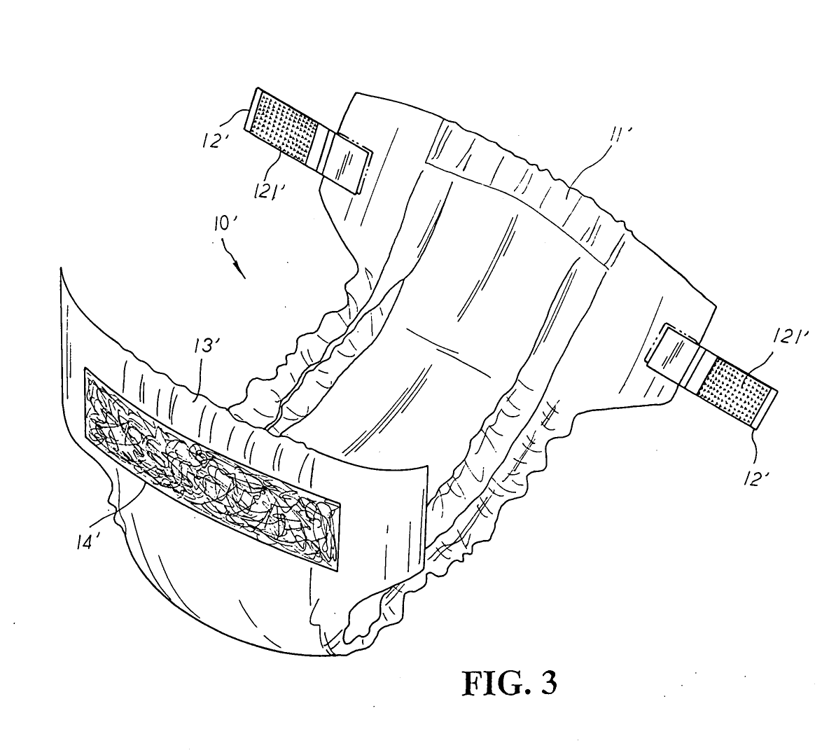

### DETAILED DESCRIPTION OF THE PREFERRED EMBODIMENTS

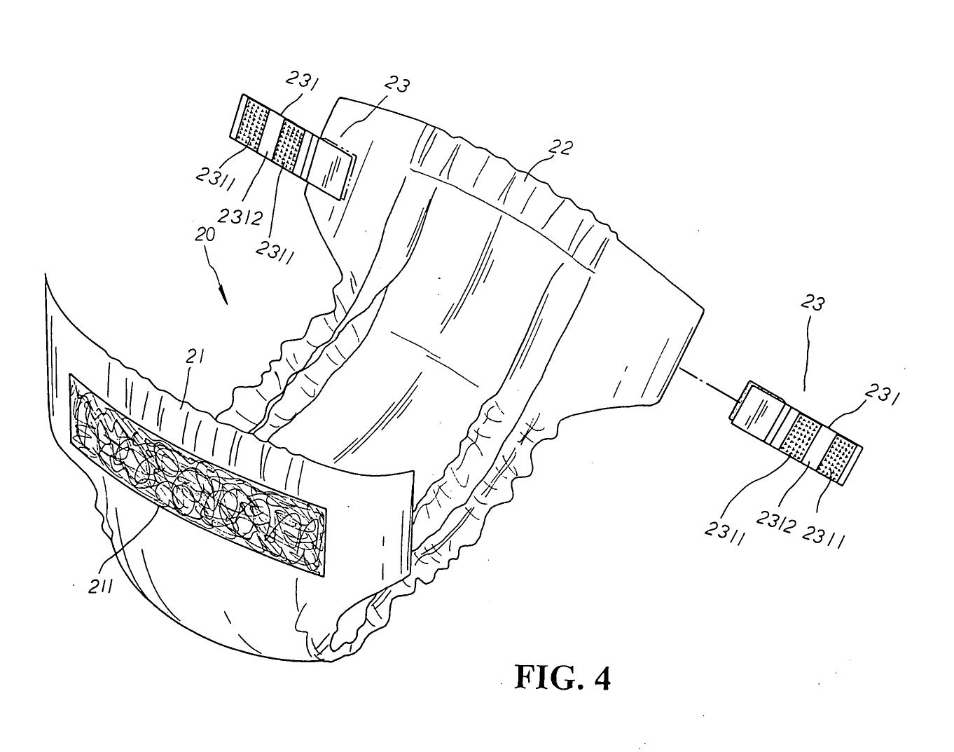

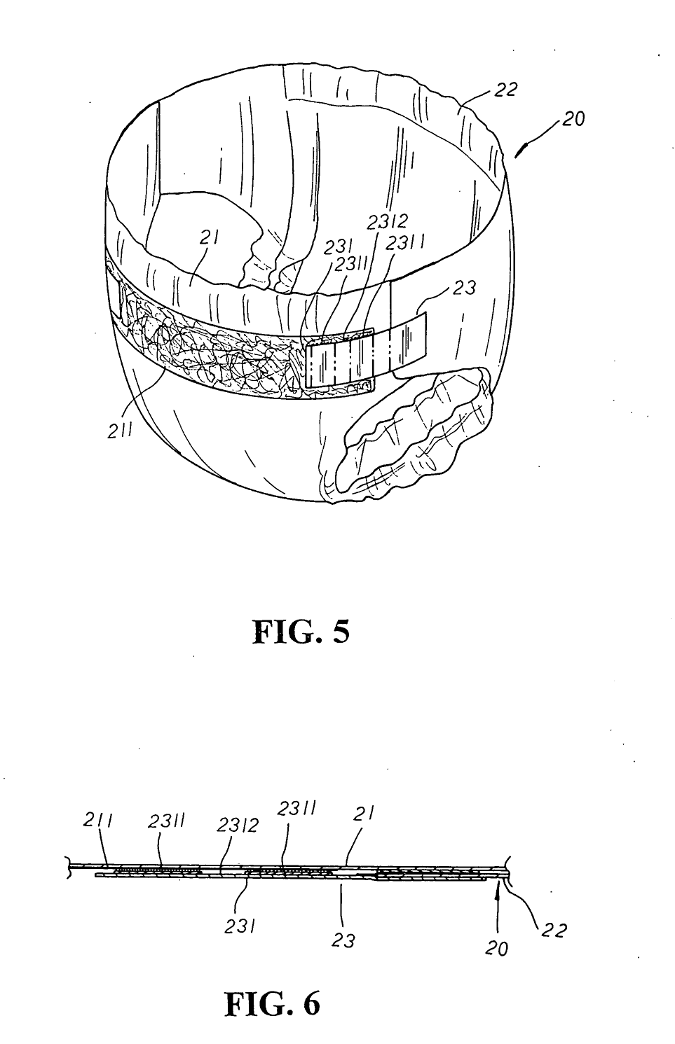

Please refer to FIGS. 4 to 6 inclusive. The present invention is related to a buckling device for disposable articles, including a disposable article 20 made up of a front piece 21 with a fleece sticker 211 disposed thereon, and a rear piece 22 having adjusting buckle belts 23 attached at both lateral edges thereon to be mutually fastened to the fleece sticker 211 of the front piece 21 thereby. The adjusting buckle belt 23 has a buckling section 231 made up of a plurality of jump-type fastening facets 2311 and flexible soft facets 2312 alternatively arranged at the inner lateral side of the buckling section 231 thereon to keep the proper softness of the buckling section 231 thereof. In buckling engagement, the fastening facets 2311 of the buckling sections 231 thereof are firmly attached to the fleece sticker 211 thereof respectively in a jump-type manner with the flexible soft facets 2312 alternatively dividing, the fastening facets thereof as shown in FIG. 6. Thus, both the front and rear parts of the buckling sections 231 thereof can be precisely buckled up with the fleece sticker 211 thereof to effect the best buckle engagement thereof, preventing the buckling sections 231 thereof from being twisted or torn apart easily by toddlers in great and active movements. Besides, via the flexible soft facets 2312 alternatively arranged with the fastening facets 2311 thereof, the buckling sections 231 are maintained in proper softness as a whole, permitting a comfortable feeling to the toddlers by the belly skin while walking around with the disposable article 20 so as to achieve the best using condition of the disposable article 20 thereof.

1. (canceled)

2. A buckling device for a disposable article comprising:
a) a fleece sticker located on a front of the disposable article; and
b) two adjustment belts removably connected to the fleece sticker, one of the two adjustment belts is located on each of two opposing sides of a rear section of the disposable article, each of the two adjustment belts having a buckling section having:
i) a plurality of fastening fasteners, each of the plurality of fastening fasteners extending from a first edge to a second edge of the buckling section; and
ii) at least one flexible soft facet, one of the at least one flexible soft facet is located between and separating each set of two adjacent fastening fasteners of the plurality of fastening fasteners.
